(19) United States
(12) Patent Application Publication
(10) Pub. No.: US 2022/0016292 A1
(43) Pub. Date: Jan. 20, 2022

(54) Antiviral Compound with UV Reflectivity

(71) Applicant: ViruShield, Inc., San Diego, CA (US)

(72) Inventors: Kenneth Boblak, Roseville, CA (US); Carl Savoia, Columbus, OH (US); Jue-Hyun Lee, Sacramento, CA (US); Vijaya K. Kuruganti, Fairfax, VA (US); Crew Winkler, Folsom, CA (US); Briant Benson, Placerville (YS); Michael A. Dobbins, Newcastle, CA (US); David A. Ottman, Carmichael, CA (US)

(21) Appl. No.: 17/381,066

(22) Filed: Jul. 20, 2021

Related U.S. Application Data
(60) Provisional application No. 63/053,911, filed on Jul. 20, 2020.
(60) Provisional application No. 63/088,923, filed on Oct. 7, 2020.
(60) Provisional application No. 63/211,229, filed on Jun. 16, 2021.

(51) Int. Cl. A61L 2/26; A61L 2/10; A61L 2/18; B65D 83/14

(52) U.S. Cl. CPC A61L 2/26; A61L 2/10; A61L 2/18; A61L 2101/02; A61L 2202/15; A61L 2202/11; B65D 83/14

(57) ABSTRACT

A dispenser comprising a dispensing device containing a dispensing device solution, the dispensing device solution comprising C₂₀H₂₉NO₇Si, the dispensing device solution having antiviral properties and emitting or reflecting UV light, wherein the dispensing device solution may be used in combination with antimicrobial products. wherein the dispensing device solution further comprises polymerizable UV absorbing and fluorescing elements, wherein the polymerizable UV absorbing and fluorescing elements are biodegradable, water soluble and copolymerize into hydrogel polymers, the dispensing device further comprising a dispensing unit. To verify proper application, a UV light source may directed to the dispensed solution.

### RELATED APPLICATIONS

This application claims the benefit of U.S. provisional patent application 63/053,911 filed Jul. 20, 2020, and further claims the benefit of U.S. provisional patent application 63/088,923, filed Oct. 7, 2020, and further claims the benefit of U.S. provisional patent application 63/211,229, filed Jun. 16, 2021. The disclosures of these provisional applications are incorporated herein as if set out in full.

### TECHNICAL FIELD OF THE DISCLOSURE

The present embodiment relates in general to liquids, foams, sprays and their dispensers. In particular, the present invention relates to a liquid, foam, or spray with anti-viral properties and ultraviolet light reflectivity.

### BACKGROUND OF THE DISCLOSURE

It has long been desirable to eliminate or at least to control odors that result from various activities and/or which are associated with certain objects and places. In addition, disinfecting the potentially contaminated sources associated with said odors is desirable as well. Garbage cans, dumpsters, trash bags, dirty clothes hampers, and a wide variety of other articles used in homes, commercial settings and industry are several common sources of unpleasant odors. Over the years, excellent cleaning products and disinfectants, including soaps and detergents containing antimicrobial agents, have been developed. However, in some cases cleaning is not an effective or practical means for odor control.

Odor management compositions known in the art can be divided into three categories which are based on their functionality. These categories of odor management compositions are defined as odor masking compositions (which masks odors through the use of fragrances or perfumes), deodorizing/sanitizing compositions, which bind to odors or eliminate the microorganisms that are responsible for the production of said odors, and combination odor masking and deodorizing/sanitizing compositions (which bind to odors and eliminate the microorganisms responsible for the production of said odors, as well as introducing a perfume or fragrance). Odor masking compositions primarily function by providing a large quantity of a perfume or fragrance that overwhelms the senses, masking odors without removing or modifying the source of said odor. Deodorizing/sanitizing compositions function by containing active agents that function in a deodorizing and antimicrobial capacity. The deodorizing agents chemically bind to existing odors thereby deactivating them, while the antimicrobial agents are responsible for eliminating the microorganisms responsible for the production of said odors. Combination odor masking and deodorizing/sanitizing compositions are provided with both a deodorizing/sanitizing agent and an odor masking composition that eliminates the source of a particular odor while providing an additional fragrance or perfume to the area of application. Of these odor management compositions, deodorizing/sanitizing compositions are of particular interest due to their various applications and incorporation into new and existing odor management systems.

Deodorizing/sanitizing compositions known in the art can be formulated using a plurality of active deodorizing/sanitizing agents. One of these active sanitizing agents includes sodium tetraborate decahydrate, commonly known as “borax.” Borax is a boron salt that has the chemical formula Na₂[B₄O₅(OH)₄]₈H₂O in solution. Borax is able to function as a deodorizing/sanitizing agent as a result of its co-complexing ability that enables it to stably bind with various substances forming complex ions. The ability to form complex ions enables borax to function as a deodorizing agent but additionally grants it antimicrobial properties. These antimicrobial properties are a result of the borax formed complex ions inhibiting key metabolic pathways of several microorganisms.

Colloidal silver is another active deodorizing/sanitizing agent. Colloidal silver is a metallic silver nanoparticle formed after ionization of silver or as a result of a chemical reaction which synthesizes zero-valent silver from mono-valent silver cations. The zero-valent silver cations that are formed disperse in a colloidal suspension, wherein the colloidal suspension provides silver nanoparticles separated by between 10 nanometers (nm) and 100 nanometers (nm). Through this unique arrangement, silver nanoparticles have unique optical, electrical and thermal properties, in part due to significant surface area to volume ratio. The colloidal dispersal of the silver nanoparticles grants a solution with silver nanoparticles with deodorizing and antimicrobial properties. The deodorizing properties are provided by the ability of the silver nanoparticles to react with substances more frequently due to the surface area to volume ratio. The antimicrobial properties are provided by the ability of the silver nanoparticles to inhibit aerobic metabolism in various microorganisms.

Despite the use of various deodorizing/sanitizing compositions, infectious diseases continue to be the third leading cause of death in the United States and worldwide. Healthcare-associated infections (HAIs) continue to be one of the world's most pressing and expensive healthcare problems. Soiled hard and soft surfaces, in addition to airborne contaminants, play an important role in transmission of infections, and are responsible for much of the documented outbreaks of healthcare-associated infections. Cross-infections are not only the main causes of morbidity and mortality in hospitals, but they also increase hospital stays and costs. The rates of nosocomial infections, especially by those caused by antibiotic resistant bacteria, are increasing alarmingly over the globe.

A central factor for transmission of infectious agents is the ability of microorganisms to survive on environmental surfaces and in the air. It has been well-established that many infectious agents can survive for a long period of time in the environment. For example, on various hospital surfaces and air systems, gram-positive bacteria (vancomycin-sensitive and -resistant Enterococci and methicillin-sensitive and -resistant Staphylococci) survive for at least one day, and some survive for more than 90 days; gram-negative bacteria (including Pseudomonas aeruginosa, Escherichia coli, Klebsiella pneumoniae, Serratia marcescens, Proteus mirabilis, Acinetobacter species, and Enterobacter species) survive from 2 hours to more than 60 days; medically important fungi (Candida spp., Aspergillus spp., Fusarium spp., Mucor spp., and Paecilomyces spp.) survive for days to weeks; and viruses (parainfluenza viruses, influenza A and B viruses, respiratory syncytial viruses, human enteric viruses and SARS coronavirus) can survive for hours to days. For a surface example, a hospital outbreak of Methicillin-resistant Staphylococcus aureus (MRSA) was directly linked in one study to a stretcher and a handheld shower; a Pseudomonas aeruginosa outbreak in a hematology-oncology unit was caused by contaminated surface cleaning equipment; and a norovirus outbreak at a long-term-care facility was associated with contaminated surfaces of case-residents' rooms, dining room tables, and elevator buttons. Recent studies showed that patients harboring multidrug-resistant bacteria such as MRSA and Vancomycin-Resistant Enterococci (VRE) could heavily contaminate their surrounding environment, and the contaminated surfaces could significantly increase the risk of transmission to subsequent occupants.

Given the proliferation of infectious pathogens and especially in the wake of Covid-19 in 2020, the development of antimicrobial surfaces and airborne foggers that effectively inactivate pathogens, odor-causing microorganisms and prevent biofilm formation has become an urgent issue. However, successful examples are still few and limited in scope. Quinolones are known to exhibit potent durable antimicrobial and antiviral properties against microorganisms. Additionally, they can prevent or minimize noxious odors by inactivating upon contact with microorganisms. In various studies, quinolones have been shown to inactivate and/or dampen malodorous products of microbes and the enzymes that produce them. For example, quinolones have been shown to abate malodorous vapors that result from the decomposition of organic matter in bodily wastes. In other instances, increased attention has been granted to aerosolized sprays, novel reaction chemistries enhancing quinolone activity, and devices more readily disperse or visualize disinfectants known in the art.

### SUMMARY

This application relates to antiviral, antibacterial, disinfecting and deodorizing compounds, synthetic routes to their synthesis, and related dispensers such as foggers and attached UV light sources. In some embodiments a dispensing device may include a dispensing device solution comprising C₂₀H₂₉NO₇Si, the dispensing device solution having antiviral properties, reflecting UV light, and is usable in combination with antimicrobial products. In addition, the dispensing device solution may include polymerizable UV absorbing and fluorescing elements that are biodegradable, water soluble and copolymerize into hydrogel polymers.

In some embodiments, the dispensers include dispensing units providing an aerosolized spray projected from a cannister or hose operably attached to the dispensing unit. Further, the dispensing device solution may have antibacterial properties such as those provided by titanium dioxide (i.e., mediated by hydroxyl radical formation). In some embodiments, the UV absorbing and fluorescing elements include 2-phenyl benzotriazole having a polymerizable acrylic group. In other embodiments, bonding of two different UV absorbing compounds with different UV absorbing spectra is permitted by the synthetic reaction chemistry provided.

In some embodiments, the dispensing unit solution may further include Chlorine dioxide, Quaternary ammonium Citric acid, Thymol Dodecylbenzenesulfonic acid, Lactic acid, Ethyl alcohol, Quaternary Ammonium, Glycolic acid, Hydrochloric acid, Hydrogen peroxide, Hydrogen peroxide, Ammonium carbonate, Ammonium bicarbonate, Hydrogen peroxide, Octanoic acid, Peroxyacetic acid, Peroxyoctanoic acid, Peroxyacetic acid, Hydrogen peroxide, Silver Hypochloric acid, Hypochlorous acid, Isopropyl alcohol, Quaternary ammonium, L-Lactic acid, Lactic acid, Octanoic acid, Peracetic acid, Phenolic, Ethanol, Potassium peroxymonosulfate, Sodium chloride, Quaternary ammonium, Isopropanol Quaternary ammonium, Isopropanol, Glutaraldehyde, Sodium carbonate, Peroxyhydrate, Silver ion, Sodium chloride, Sodium chlorite, Sodium dichloroisocyanurate dihydrate, Sodium dichloro-S-triazinetrione, Sodium dichloroisocyanurate, Sodium hypochlorite, Sodium carbonate, Thymol, and/or Triethylene glycol.

In other embodiments, antiviral disinfecting solutions may include at least one quinolone solution, wherein the quinolone solution reflects UV light. In some embodiments, the antiviral disinfecting solution further includes polymerizable UV absorbing and fluorescing elements. In other embodiments, the antiviral disinfecting solution has antibacterial properties wherein titanium dioxide provides the antibacterial mechanism for the antibacterial properties.

In some embodiments, the polymerizable UV absorbing and fluorescing elements are biodegradable, water soluble and easily copolymerize into hydrogel polymers. Notably, the antibacterial mechanism of titanium dioxide is induced by UV wavelengths in the range of 240 nm to 280 nm. In some embodiments, the UV absorbing elements include Para Amino Benzoic Acid (PABA), Benzophenone-1, and/or Benzotriazole. In some embodiments, the antiviral disinfecting solution further includes polymerizable UV absorbing and fluorescing elements, wherein benzotriazoles are used to copolymerize the UV absorbing and fluorescing elements into hydrogel polymers.

As is known in the art, hydrogels are desirable for use in variable polymer surfaces, however because of their hydrophilic nature and expanded structures, it has been difficult to incorporate UV absorbing solutions into hydrogels. Prior art UV absorbers are generally hydrophobic and have limited solubility in hydrogels. Due in part to this limited solubility, it has been difficult to copolymerize UV absorbers with the type of hydrogel-forming monomers amenable to use as disinfectants.

It is a first objective of the present invention to provide a means of copolymerizing UV absorbers with the type of hydrogel-forming monomers amenable to use as disinfectants.

It is a second objective of the present invention to provide a novel synthetic route to the manufacture of various antiviral quinolone solutions and conjugate UV absorbing and reflecting solutions.

It is another objective of the invention to provide a reliable means of dispensing or spraying an aersolizable antiviral and UV reflective solution.

It is another objective of the invention to provide users a means of tracking the surfaces they spray with a given disinfecting solution via the use of a UV light source.

It is another objective of the invention to facilitate the eliminate of noxious odors in both public and private spaces.

It is yet another objective of the invention to achieve antiviral and antibacterial effects using a means of action that viruses and bacteria cannot easy mutate and evolve around, such as the use of hydroxyl radicals.

These and other advantages and features of the present invention are described with specificity so as to make the present invention understandable to one of ordinary skill in the art. The foregoing and other objects and features of the disclosure will become more apparent from the following detailed description, which proceeds with reference to the accompanying figures.

### BRIEF DESCRIPTION OF THE FIGURES

In order to enhance clarity and improve understanding of the various elements and embodiments of the invention, elements in the figures have not necessarily been drawn to scale. Furthermore, elements that are known to be common and well understood to those in the industry are not depicted in order to provide a clear view of the various embodiments of the invention. Thus, the drawings are generalized in form in the interest of clarity and concision.

FIG. 1A shows the UV screener (or “UV absorbing solution”) Para Amino Benzoic Acid (PABA) in accordance with one embodiment of the present invention;

FIG. 1B shows the UV screener Benzophenone-1 in accordance with one embodiment of the present invention;

FIG. 1C shows the UV screener Benzotriazole in accordance with one embodiment of the present invention;

FIG. 2 shows exemplar antiviral disinfecting solutions (i.e., dispensing device solution) including quinolone scaffold compound I. and quinolone scaffold compound II. in accordance with one embodiment of the present invention;

FIG. 3 shows the route to synthesis of antiviral disinfecting solution components quinolone compound A and quinolone compound B in accordance with one embodiment of the present invention;

FIG. 4 shows a cartoon depiction of the final product tagged with photoluminescent monomers according to one embodiment of the present invention;

FIG. 5 shows the route to synthesis of antiviral disinfecting solution components quinolone major product I and quinolone major product II in accordance with one embodiment of the present invention;

FIG. 6A shows a cartoon depiction of self-assembled siloxane polymer admixed with photoluminescent oligomer tags in accordance with one embodiment of the present invention; and

FIG. 6B shows a cartoon depiction of self-assembled siloxane polymer admixed with photoluminescent terminating group oligomer tags in accordance with one embodiment of the present invention.

### DETAILED DESCRIPTION OF THE EMBODIMENTS

In the following discussion that addresses a number of embodiments and applications of the present invention, it is to be understood that other embodiments may be utilized, and changes may be made without departing from the scope of the present invention.

Various inventive features are described below that may each be used independently of one another or in combination with other features. However, any single inventive feature may not address any of the problems discussed above or only address one of the problems discussed above. Further, one or more of the problems discussed above may not be fully addressed by any of the features described below.

The present embodiment relates in general to an antimicrobial, antiviral antibacterial, anti-mold, anti-odor, anti-Volatile Organic Compounds (“VOCs”) and/or antiviral disinfecting and deodorizing solution or mixture that is utilized in the form of a spray, fog, bomb or wipe that may be applied to all types of interior spaces, exterior spaces, surfaces, items, individuals, and objects to disinfect, sanitize, or otherwise cleanse the object, the mixture further including ultraviolet lighting tracing or detection properties. The mixture is preferably a liquid, foam, spray or wipe, and in particular a liquid, foam, spray or wipe with anti-viral properties and ultraviolet light reflectivity. In various embodiments, compound or solution is may be referred to as “the dispensing device solution” or the “antiviral disinfecting compound”.

In a preferred embodiment of the invention, the antiviral compound with ultraviolet light reflectivity includes a dispensing unit, the dispensing unit capable of dispensing the antiviral disinfecting compound in a variety of form. In some embodiments, the dispensing unit takes the form of an aerosolized spray. The spray may be projected from a cannister, or hose operably attached to the dispensing unit, and in a preferred embodiment takes the form of a fogger, or what is otherwise commonly known as a “bug bomb”. In this embodiment, the cannister fills an area with the antiviral compound. One such compound is described in U.S. Pat. No. 10,028,482, although other compounds may be used, including but not limited to soap, detergents, disinfectants, alcohol, bleaches, along with Chlorine dioxide, Chlorine dioxide, quaternary ammonium silane salts, Citric acid, Thymol Dodecylbenzenesulfonic acid, Lactic acid, Ethyl alcohol, Quaternary Ammonium, Glycolic acid, Hydrochloric acid, Hydrogen peroxide, Hydrogen peroxide, Ammonium carbonate, Ammonium bicarbonate, Hydrogen peroxide, Octanoic acid, Peroxyacetic acid, Peroxyoctanoic acid, Peroxyacetic acid, Hydrogen peroxide, Silver Hypochloric acid, Hypochlorous acid, Isopropyl alcohol, Quaternary ammonium, L-Lactic acid, Lactic acid, Octanoic acid, Peracetic acid, Phenolic, Ethanol, Potassium peroxymonosulfate, Sodium chloride, Quaternary ammonium, Isopropanol Quaternary ammonium, Isopropanol, Glutaraldehyde, Sodium carbonate, Peroxyhydrate, Silver ion, Sodium chloride, Sodium chlorite, Sodium dichloroisocyanurate dihydrate, Sodium dichloro-S-triazinetrione, Sodium dichloroisocyanurate, Sodium hypochlorite, Sodium carbonate, Thymol, and Triethylene glycol, among others. In still further embodiments, the material is a titanium dioxide. In some embodiments, the material is one of the aforementioned antiviral components that has been modified to reflect of fluoresce ultraviolet light, and in other embodiments, a separate ultraviolet light reflecting compound is associated with or included with the antiviral compound, which is added to the mix as a tracer.

In some embodiments, the compound includes an alcohol base or an ammonia chloride base, but with the additive nano silver, copper, or titanium or titanium dioxide. Notably, the antiviral and antibacterial mechanisms involving titanium dioxide are well known in the art yet uniquely adapted to the present invention. Specifically, in use when UV light becomes incident upon the titanium dioxide surface electrons are released, said electrons interacting with water molecules in the air, thereby breaking said water molecules into hydroxyl radicals, which are highly reactive. Mechanistically, said reactive molecules may break down pollutants and other harmful molecules in the air during use. In addition, UV LED lights may be used to activate said titanium dioxide surface. Said UV lights preferably emit light within a range of from ˜240 nm to ˜280 nm wavelength, and in some embodiments are powered by a battery connected to a voltage regulator, and a circuit connected between said voltage regulator and said UV LED lights for providing pulsed current thereto. An optional solar cell is disclosed for powering the UV LED lights directly or by recharging the battery, and the solar cell may be mounted atop a visor in some embodiments.

In all cases, the dispensed or sprayed material reflects or fluoresces under ultraviolet light. Black light paint, or black light fluorescent paint is luminous coloring that glows under light in the ultraviolet segment of the electromagnetic spectrum. Preferably, the material is not colorful under ordinary light. In a preferred use, a user may spray an area with the antiviral compound, which would be undetectable unless exposed to an ultraviolet light source. This allows the user to verify that the entire area, object, or individual is properly coated. Preferably, under daylight or normal light conditions, the sprayed object, material, area, or individual will not be vibrant in color, but under ultraviolet light, with little or no visible light also present, the treated area becomes readily apparent. The system disclosed herein thus also includes a related ultraviolet, or black light source, which allows the user to confirm the anti-viral or other compound has adequately coated the set area, object, or individual. The user may then verify that he or she has adequately coated whatever object(s) required coating by detecting the reflected UV light.

As described above, polymerizable UV absorbing and reflecting elements or compounds may be utilized for producing various structures and may be integrated into various lipidic solutions. For example, para-aminobenzoic acid (PABA) 12, benzophenone, benzotriazole 16, and/or substituted derivatives thereof may be utilized as UV absorbing moieties integrated into various cleansing and hand-sanitizing solutions. Further, 2-phenyl benzotriazole compounds having a polymerizable acrylic group may be used to produce variable polymer surfaces allowing dispenser bottles and other structural components themselves to contain the UV-absorptive and fluorescent properties described herein. This later UV absorption technology has been applied primarily to rigid, gas permeable variable polymer surfaces in the past; most commercially available soft variable polymer surfaces do not contain UV absorbers.

As is known in the art, hydrogels are desirable for use in variable polymer surfaces, however because of their hydrophilic nature and expanded structures, it has been difficult to incorporate UV absorbing compounds into hydrogels. Prior art UV absorbers are generally hydrophobic and have limited solubility in hydrogels. Due in part to this limited solubility, it has been difficult to copolymerize UV absorbers with the type of hydrogel-forming monomers amenable to use as disinfectants and the like. UV absorbers are preferably copolymerized or covalently bonded to the matrix, rather than physically entrapped within the hydrogel, to prevent the absorber from being leached out of the UV absorbing hydrogel when the hydrogel is in an aqueous environment. In some embodiments, benzotriazoles 16 are utilized to address these issues and to easily copolymerize into hydrogel polymers. Use of said benzotriazoles 16 ameliorates problems related to both incorporation of UV absorbers into hydrogels and those related to hydrolytic stability. As described above, UV absorbers have the required characteristics of absorption between 300-400 nm, these compounds have traditionally been difficult to synthesize.

In some embodiments, benzotriazoles 16 absorb UV light at the more energetic end of the UV spectrum and have favorable chemistries for integration with secondary fluorescent compounds. These benzotriazoles 16 copolymerize well into hydrogel polymers without the problems of leaching. Also, effective amounts of benzotriazoles 16 incorporated into surface polymers and hydrogels do not negatively affect the fluorescent properties of the polymer. In addition, these compounds have a higher absorptive cut-off, up to 400 nm, to block more light in the UVA range, thereby minimizing user skin and eye exposure to high energy wavelength light.

In some embodiments, the present invention includes bonding two different UV absorbing compounds that each have a different UV absorbing spectra. These compounds may them be applied as a coating onto a polymeric surface, may be conjugated to a water-soluble fluorophore, or may be conjugated to a biodegradable compound such as a halo imidazolidinone. The UV absorbers in this embodiment are adapted to provide the broadest UV absorption range possible, while providing chemistries adaptable to conjugation with secondary compounds. Additional UV absorbing agents may include p-aminobenzoic acid and a benzotriazole 16, or p-aminobenzoic acid and a benzophenone. These later UV absorbing compounds may only be used in a one-to-one mole ratio, so optimization of the UV absorption spectra is somewhat limited.

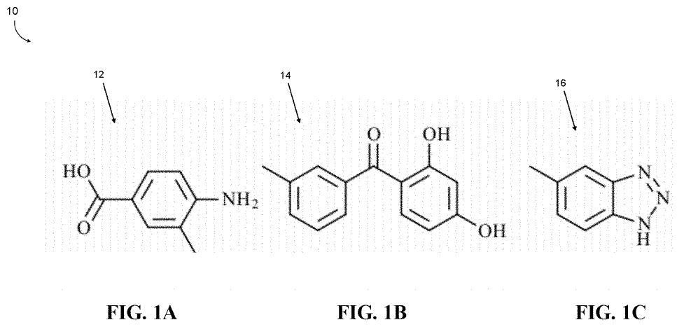

In some embodiments, the present invention overcomes UV absorption and conjugation obstacles known in the art by including variable polymers and/or water soluble fluorescent compounds formed by incorporating a combination of a first ultraviolet absorber, a second ultraviolet absorber, a biodegradable compound, and a conjugated fluorophore wherein the first UV absorber is a benzotriazole 16, a PABA moiety 12, or a benzophenone-1 moiety. In some embodiments, said water-soluble compounds are adapted to use with any sanitizer, any soap, and/or any antiviral compound that is itself partially lipophilic and adaptable to the herein described conjugation chemistries. This aspect of the invention allows a user to chemically tag soaps, medicides, deodorizers, disinfectants, anti-bacterials, and/or antiviral chemicals such that they fluoresce under a UV light. This permits a user to wash their hands and then with a black light, or alternate UV light, to observe where they coated their hands with the disinfecting compounds/solutions, and where they might have failed to coat their hands. As shown in FIG. 1, in some embodiments Para Amino Benzoic Acid (PABA) 12, Benzophenone-1 14, and/or Benzotriazole 16 are used as UV screeners (i.e., UV absorbing compounds) 10, thereby facilitating detection of coating efficacies.

In other embodiments, as described above, said UV absorptive compounds may be used in combination with fluorescent compounds. Conjugation between said fluorescent compounds may be chemical, as described, or biochemical. In the latter case, absorption of UV light may trigger a conformational change in a biopolymer, such as an antibody. Furthering the antibody example, the triggering of said conformation change in a primary antibody is transmitted down the FC region of the antibody, ultimately inducing a conformational change at the terminus of said FC region, thereby exposing an integrated enzyme in the primary antibody, the integrated enzyme subsequently activating fluorescence in a secondary antibody upon binding of the variable region of the secondary antibody. In an alternative chemical example, any of a variety of benzene ring systems may capture incident UV light, causing a conformational change within a small molecule, resulting in a color change (spectral shift) resulting from variable absorptive capacities of the small molecule in the visual spectra.

In some embodiments, conjugated biodegradable compounds include EPA-approved halohydantoins and imidazolidinones (for example, the compound Medecide) that have been rigorously tested to ensure environmental, ecological, and consumer safety. For example, the various halohydantoins utilized herein have been screened to limit harmful dermatological and chemical interactions that may pose a harm to users. Applications in food processing, food and beverage containers, water treatment, hard surfaces, pools and spas, and on eggs, fruit and vegetables has also been contemplated. Indeed, there is an estimated annual consumption of halohydantoins in the U.S. of >16 million kgs/year. In other embodiments, uses of Medecide that are not biocidal are also contemplated as described in U.S. Pat. No. 10,028,482 (2018), and 10,512,705 (2019). These compounds may be utilized as stabilizers, surface binding agents, dispersants without posing a hazard to users, waste streams, and the like.

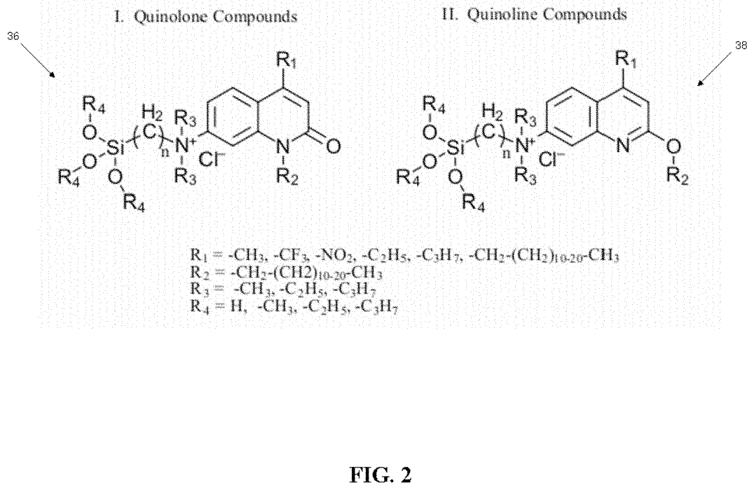

As described above, the present invention contemplates a variety of UV absorbers (i.e., UV screeners) 10 and UV stabilizers including those commonly used in sunscreen lotions and other personal care applications. As shown in FIG. 1, Para Amino Benzoic Acid (PABA) 12, Benzophenone-1 14, and/or Benzotriazole may also be used as potential UV screeners 10 to detect coating efficacies. As shown in FIG. 2, the present invention contemplates a variety of quinolone compound scaffolds including quinolone scaffold compound I. 36 and quinolone scaffold compound II. 38. These quinolone compound scaffold include substitutable R groups R₁ through R₄ in one embodiment of the present invention. Various R groups are contemplated beyond those shown in FIG. 2, including those with R groups of various length and carbon content.

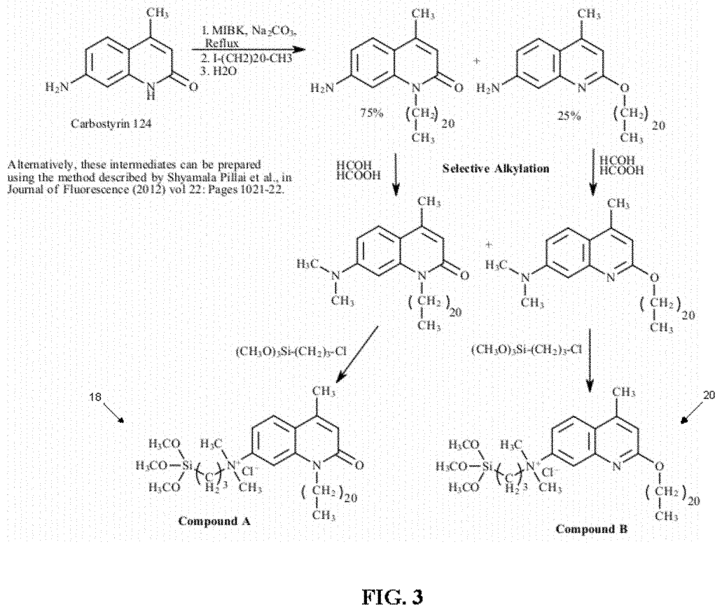

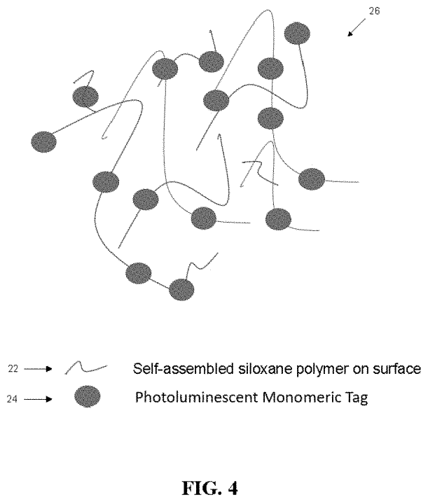

As described below, quinolone major product I. 42, quinolone major product II. 44, quinolone compound A 18, and quinolone compound B 20 represent different quinolone compounds used to formulate various embodiments of the dispensing device solution (also referred to herein as, “antiviral disinfecting solution”) described herein. As illustrated in FIG. 3, a novel synthetic route to produce fluorescent antimicrobial compound A 18 and compound B 20 is provided. As shown in FIG. 3, synthetic route initiates with Carbostyrin 124 and, after selective alkylation, compound A 18 and compound B 20 are produced. This approach relies on improvements to chemical steps known in the art as described below (See method described by Shyamala Pillai et al. in Journal of Fluorescence (2012) vol 22: Pages 1021-22). FIG. 4 shows a cartoon depiction of the final product (self-assembled siloxane polymer) 22 tagged with photoluminescent monomeric tags 24 to form a self-assembled siloxane polymer/monomeric tag admixture 26 according to one embodiment of the present invention. Notably, upon ejection from the dispenser, it is the below-described dispensing device solution that forms a self-assembled siloxane polymer and monomeric tag admixture 26, wherein the fluorescence tag self assembles with the other monomer to form a self-assembled macromer coating.

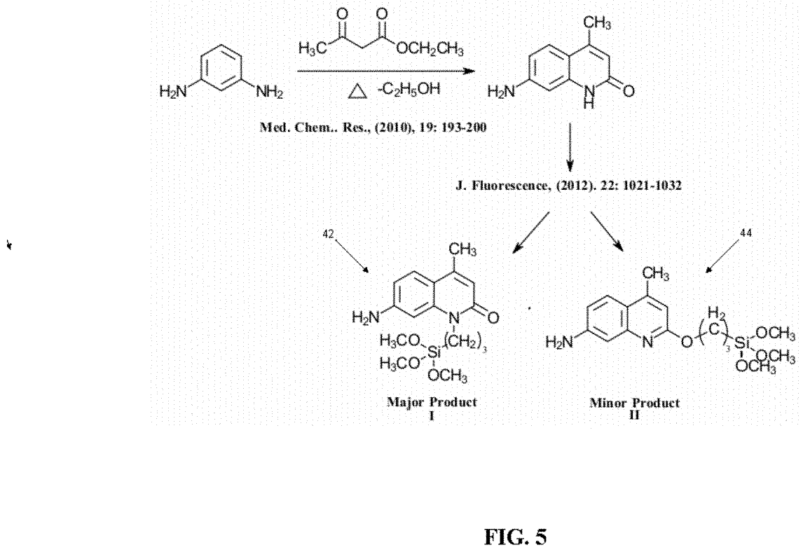

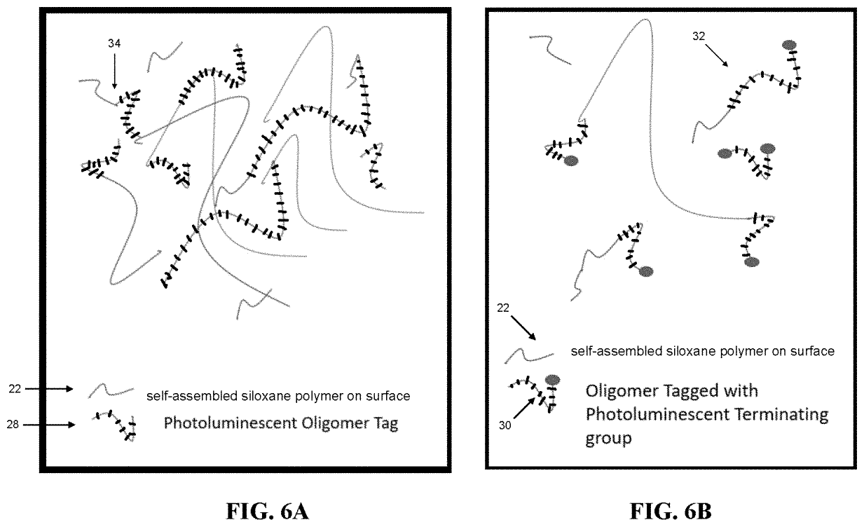

As illustrated in FIG. 5, a novel synthetic route to produce major product I 42 and minor product II 44 is further provided by the present invention. This approach relies improvements to chemical steps known in the art as described below (See also Med. Chem., Res., (2010), 19:193-200; J. Fluorescence, (2012). 22: 1021-1032). FIG. 6A shows a cartoon depiction of self-assembled siloxane polymer 22 tagged with photoluminescent oligomer tags 28 to form a self-assembled siloxane polymer/photoluminescent oligomer tag admixture 34 according to one embodiment of the present invention. Similarly, FIG. 6B shows a cartoon depiction of self-assembled siloxane polymer 22 tagged with photoluminescent terminating groups 28 to form a self-assembled siloxane polymer/photoluminescent terminating group admixture 32 according to one embodiment of the present invention. Notably, in use it is the antiviral disinfecting solution itself that may form an photoluminescent oligomer tag and self-assembled siloxane polymer admixture 34 or photoluminescent terminating group and self-assembled siloxane polymer admixture 32.

In another embodiment, the system may be used in connection with widely used antimicrobial products that differ in compositions ranging from Alkalies, Alcohols, Aldehydes, Chlorine Active Compounds, lodophores, Phenolics, Peroxygens, Quaternary Ammonium Compounds (QACs) and combinations thereof. Although several of the antimicrobial compositions are effective such as Ethyl Alcohol but their duration of effectiveness is limited for short period of times. In recent years, many antimicrobial compositions have been developed that contain both short term effective antimicrobials in combination with long term effective antimicrobials. Of particular interest are quaternary silyl ammonium chloride compounds that offer long term antimicrobial activity in combination antimicrobials with short term antimicrobial activity such as Alcohols and Benzalkonium Chloride compounds or BACs.

In some embodiments this product is combined with the disinfectant Monofil D (MD), a quaternary ammonium salt with trihydroxysilyl (THS) functionality. These antimicrobials can be labeled with a fluorescent tag. When labeled with a fluorescent tag these antimicrobials will be referred to as the “parent molecule” or “parent compound”. The parent compound can be a small molecule, monomer, oligomer or polymer. In some embodiments the parent compound is intended to covalently bond with monomers that contain a reactive THS functional group. THS molecules can be obtained from trialkoxysilanes through hydrolysis in aqueous media with appropriate catalysts known by those skilled in the art. This parent molecule will be referred to as DNC-1. When the disinfectant is applied to a surface the individual monomers condense to form a polymeric, macromolecular coating that is telechelic. We will call this the target molecule. This means that it is a capable of further polymerization or reaction at its end groups. This property allows one to tag these polymers with any photoluminescent molecule that has a hydroxy group, amine group, are THS monomers, are oligomers or polymers with the siloxane backbone and are not end capped. When assembled on the surface, DNC-1 and Monofoil D covalently bond and exhibit antimicrobial and fluorescent behavior. These behaviors increase or decrease dependently on one another. This allows for proof of initial coverage of the product as well as monitoring mechanical removal of the product. Listed below are the details of Methods A, B, C and D, which are used to achieve the above. Most preferably, Method D is used to achieve this goal.

### Method A: Direct Synthesis of Fluorescent Quaternary Ammonium Salt Silanes:

Substituted Quinolone and Quinoline compounds are widely used in Ultraviolet fluorescent labeling, and it has been demonstrated that the absorption and emission spectra of these compounds can be altered depending upon the substituents on Quinolone and Quinoline rings as shown in part by the quinolone I. 36 and quinolone II. 38 structures shown in FIG. 2. As shown in FIG. 4, self-assembled siloxane polymer and photoluminescent monomeric tags may also be admixed in solution to form a heterogenous self-assembled siloxane polymer/monomeric tag admixture 26. The current invention describes compositions that offer long term antimicrobial activity and that also benefit from UV-inducible fluorescence activity. Since the fluorescent moiety is chemically bound to structural groups responsible for antimicrobial activity, it is possible to track the duration of antimicrobial activity using UV fluorescence in some embodiments.

Method B: Fluorescent Silane Monomers Polymerized with THS Functionalized Monomers:

In another embodiment, as shown in FIG. 5, synthetic routes to product fluorophores I and II (also referred to herein as “major product I” and minor product II″) are provided. The resultant novel UV fluorophores can be inter-polymerized with siloxane containing antimicrobial compounds based on N-Alkyl alkoxy silyl ammonium chloride and N-Alkyl hydroxy silyl ammonium chloride (e.g. Common Sense®, Monofoil D® (also known as MicroGold® Multi-Action Disinfectant Antimicrobial Spray, by MicroGold, L.L.C. FORMERLY MicroGold, Inc.), Goldshield®, Biosafe®, etc.). Once polymerized with antimicrobial compounds, these UV fluorophores can be used for tracking antimicrobial activity of those compounds. FIG. 5 illustrates the final product on the substrate. Specifically, FIG. 5 shows the synthetic route to formation of major product I and major product II.

In further embodiments, o-4-Methylcoumarinyl-n-[3-(triethoxysilyl)propyl]carbamate may be added to the composition, chemical structure C₂₀H₂₉NO₇Si and also known as 4-methyl-2-oxo-2H-chromen-7-yl 3-(triethoxysilyl)propylcarbamate. The compound, solid at room temperature and pressure may be added to MonoFoil D® at 0.1% to 2% by weight concentration to the overall volume of the final formulation including water. In an exemplary case of 1 gallon of final solution, this would require approximately 7.5 grams of C₂₀H₂₉NO₇Si in order to make 0.2% by weight solution. As a process, the solid is thoroughly mixed with the MonoFoil D® before final dilution with water to arrive at the final diluted mix. The C₂H₂₉NO₇Si will react with the water to produce hydroxy silane and ethanol. This photoluminescent molecule also has the THS functionality which allows it to self-assemble with MD when applied to the surface thereby yielding a blue or green fluorescence of the final coating when exposed to UV light.

Method C: Oligomer with Photoluminescent Monomer Assembled with Monofoil D

In another embodiment an oligomer is prepared from a photoluminescent, THS or TAS monomers, such as o-4-Methylcoumarinyl-n-[3-(triethoxysilyl)propyl]carbamate with other monomers that can be used for different functions including but not limited to: solubility, adhesion, UV absorbance, emission, color, elasticity, refractive index, conduction, insulation, etc. This is then condensed on the surface with MD to give the photoluminescent oligomer tag and monofoil polymer admixture 34 shown in FIG. 6A.

Method D: Oligomer Tagged with Photoluminescent Small Molecule Assembled with Monofoil D.

In another embodiment, an oligomer or polymer is prepared from THS monomers that can be used for different functions including but not limited to: solubility, adhesion, UV absorbance, emission, color, elasticity, refractive index, conduction, insulation, etc. After polymerization, the oligomer or polymer is further condensed with a fluorescent small molecule. This material is post-modified at a reactive, terminating hydroxy group or also along the polymer backbone through condensation with a photoluminescent small molecule. The oligomer is then applied with MD to give the photoluminescent terminating group and self-assembled siloxane polymer admixture 32 structure shown in FIG. 6B.

Regarding the Method D Procedure, the process to synthesize Eye See Clean can involve two chemical reactions. The first reaction is the hydrolysis of all silyl ether monomers or organosilanes. Preferred organosilanes include, but are not limited to those in table 1, below:

TABLE 1
3-(trimethoxysilyl)propyldimethyloctadecyl ammonium chloride,
3-(trimethoxysilyl)propylmethyldi(decyl) ammonium chloride,
3-chloropropyltrimethylsilane,
octadecyltrimethoxysilane,
2-methoxy(polyethyleneoxy)₉₋₁₂propyltrimethoxysilane,
2-methoxy(polyethyleneoxy)₆₋₉heptamethyltrisiloxane,
perfluorooctyltriethoxysilane,
(CH₃ O)₃ Si(CH₂)₃ N⁺ (CH₃)₂ C₁₈ H₃₇ Cl⁻,
(CH₃ O)₃ Si(CH₂)₃ N⁺ (CH₃)₂ C₁₈ H₃₇ Br⁻,
(CH₃ O)₃ Si(CH₂)₃ N⁺ (C₁₀ H₂₁)CH₃ Cl⁻,
(CH₃ O)₃ Si(CH₂)₃ N⁺ (C₁₀ H₂₁)CH₃ Br⁻,
(CH₃ O)₃ Si(CH₂)₃ N⁺ (CH₃)₃ Cl⁻,
(CH₃ O)₃ Si(CH₂)₃ N⁺ (CH₃)₂ C₈ H₁₇ Cl⁻,
(CH₃ O)₃ Si(CH₂)₃ N⁺ (CH₃)₂ C₁₀ H₂₁ Cl⁻,
(CH₃ O)₃ Si(CH₂)₃ N⁺ (CH₃)₂ C₁₂ H₂₅ Cl⁻,
(CH₃ O)₃ Si(CH₂)₃ N⁺ (CH₃)₂ C₁₄ H₂₉ Cl⁻,
(CH₃ O)₃ Si(CH₂)₃ N⁺ (CH₃)₂ C₁₆ H₃₃ Cl⁻,
(CH₃ O)₃ Si(CH₂)₃ N⁺ (CH₃)₂ C₂₀ H₄₁ Cl⁻,
(CH₃ O)₃ Si(CH₂)₃ N⁺ (C₄ H₉)₃ Cl⁻,
(CH₃ O)₃ Si(CH₂)₃ N⁺ (C₂ H₅)₃ Cl⁻,
(CH₃ CH₂ O)₃ Si(CH₂)₃ N⁺ (CH₃)₂ C₁₈ H₂₇ Cl⁻,
(CH₃ O)₃ Si(CH₂)₃ NHC(O)(CF₂)₆ CF₃,
(CH₃ O)₃ Si(CH₂)₃ NHC(O)(CF₂)₈ CF₃,
(CH₃ O)₃ Si(CH₂)₃ NHC(O)(CF₂)₁₀ CF₃,
(CH₃ O)₃ Si(CH₂)₃ NHC(O)(CF₂)₁₂ CF₃,
(CH₃ O)₃ Si(CH₂)₃ NHC(O)(CF₂)₁₄ CF₃,
(CH₃ O)₃ Si(CH₂)₃ NHC(O)(CF₂)₁₆ CF₃,
(CH₃ O)₃ Si(CH₂)₃ NHSO₂ (CF₂)₇ CF₃,
(CH₃ O)₃ Si(CH₂)₃ N⁺ (CH₃)₂ (CH2)₃ NHC(O)(CH2)₆ CH₃,
(CH₃ O)₃ Si(CH₂)₃ N⁺ (CH₃)₂ (CH₂)₃ NHC(O)(CH₂)₈ CH₃,
(CH₃ O)₃ Si(CH₂)₃ N⁺ (CH₃)₂ (CH₂)₃ NHC(O)(CH₂)₁₀ CH₃,
(CH₃ O)₃ Si(CH₂)₃ N⁺ (CH₃)₂ (CH₂)₃ NHC(O)(CH₂)₁₂ CH₃,
(CH₃ O)₃ Si(CH₂)₃ N⁺ (CH₃)₂ (CH₂)₃ NHC(O)(CH₂)₁₄ CH₃,
(CH₃ O)₃ Si(CH₂)₃ N⁺ (CH₃)₂ (CH₂)₃ NHC(O)(CH₂)₁₆ CH₃,
(CH₃ O)₃ Si(CH₂)₃ N⁺ (CH₃)₂ (CH₂)₃ NHC(O)(CF₂)₆ CF₃,
(CH₃ O)₃ Si(CH₂)₃ N⁺ (CH₃)₂ (CH₂)₃ NHC(O)(CF₂)₈ CF₃,
(CH₃ O)₃ Si(CH₂)₃ N⁺ (CH₃)₂ (CH₂)₃ NHC(O)(CF₂)₁₀ CF₃,
(CH₃ O)₃ Si(CH₂)₃ N⁺ (CH₃)₂ (CH₂)₃ NHC(O)(CF₂)₁₂ CF₃,
(CH₃ O)₃ Si(CH₂)₃ N⁺ (CH₃)₂ (CH₂)₃ NHC(O)(CF₂)₁₄ CF₃,
(CH₃ O)₃ Si(CH₂)₃ N⁺ (CH₃)₂ (CH₂)₃ NHC(O)(CF₂)₁₆ CF₃,
(CH₃ O)₃ Si(CH₂)₃ N⁺ (CH₃)₂ (CH₂)₃ NHSO₂ (CF₂)₇ CF₃,
(CH₃ O)₃ Si(CH₂)₃ N⁺ (CH₃)₂ (CH₂)₃ NHSO₂ (CF₂)₉ CF₃,
(CH₃ O)₃ Si(CH₂)₃ N⁺ (CH₃)₂ (CH₂)₃ NHSO₂ (CF₂)₁₁ CF₃,
(CH₃ O)₃ Si(CH₂)₃ N⁺ (CH₃)₂ (CH₂)₃ NHSO₂ (CF₂)₁₃ CF₃,
(CH₃ O)₃ Si(CH₂)₃ N⁺ (CH₃)₂ (CH₂)₃ NHSO₂ (CF₂)₁₅ CF₃,
(CH₃ O)₃ Si(CH₂)₃ N⁺ (CH₃)₂ (CH₂)₃ NHSO₂ (CF₂)₁₆ CF₃,
aminoethylaminopropyltrimethoxysilane: NH₂ (CH₂)₂ NH(CH₂)₃
Si(OCH₃)₃,
3-aminopropyltrimethoxysilane: NH₂ (CH₂)₃ Si(OCH₃)₃,
3-aminopropyltriethoxysilane: NH₂ (CH₂)₃ Si(OCH₂ CH₃)₃,
3-chloropropyltrimethoxysilane: Cl(CH₂)₃ Si(OCH₃)₃,
3-chloropropyltriethoxysilane: Cl(CH₂)₃ Si(OCH₂ CH₃)₃,
3-chloropropyltrichlorosilane: Cl(CH₂)₃ SiCl₃,
3-glycidoxypropyltrimethoxysilane: C₃ H₅ O₂ (CH₂)₃ Si(OCH₃)₃,
3-glycidoxypropyltriethoxysilane: C₃ H₅ O₂ (CH₂)₃ Si(OCH₂ CH₃)₃,
3-methacryloxypropyltrimethoxysilane: C₄ H₅ O₂ (CH₂)₃ Si(OCH₃)₃,
3-methacryloxypropyltriethoxysilane: C₄ H₅ O₂ (CH₂)₃
Si(OCH₂ CH₃)₃,
methyldichlorosilane: CH₃ SiHCl₂,
silane-modified melamine: Dow Corning Q1-6106,
sodium (trihydroxysilyl)propylmethylphosphonate:
NaO(CH₃ O)P(O)(CH₂)₃ Si(OH)₃,
trichlorosilane, SiHCl₃,
n-2-vinylbenzylamino-ethyl-3-aminopropyltrimethoxysilane HCL:
Dow Corning Z-6032,
vinyltriacetoxysilane: H₂ C═CHSi(OCOCH₃)₃,
vinyltrimethoxysilane: H₂ C═CHSi(OCH₃)₃,
vinyltriethoxysilane: H₂ C═CHSi(OCH₂ CH₃)₃,
vinyltrichlorosilane: H₂ C═CHSiCl₃,
dimethyldichlorosilane: (CH₃)₂ SiCl₂,
dimethyldimethoxysilane: (CH₃)₂ Si(OCH₃)₂,
diphenyldichlorosilane: (C₆ H₅)₂ SiCl₂,
ethyltrichlorosilane: (C₂ H₅)SiCl₃,
ethyltrimethoxysilane: (C₂ H₅)Si(OCH₃)₃,
ethyltriethoxysilane: (C₂ H₅)Si(OCH₂ CH₃)₃,
isobutyltrimethoxysilane,
n-octyltriethoxysilane,
methylphenyldichlorosilane: CH₃ (C₆ H₅)SiCl₂,
methyltrichlorosilane: CH₃ SiCl₃,
methyltrimethoxysilane: CH₃ Si(OCH₃)₃,
phenyltrichlorosilane: C₆ H₅ SiCl₃,
phenyltrimethoxysilane: C₆ H₅ Si(OCH₃)₃,
n-propyltrichlorosilane: C₃ H₇ SiCl₃,
n-propyltrimethoxysilane: C₃ H₇ Si(OCH₃)₃,
silicon tetrachloride: SiCl₄,
ClCH₂C₆H₄CH CH SiCl₃ₙ,
ClCH₂C₆H₄CH₂CH₂ Si(OCH₃)₃,
ClCH₂C₆H₄CH₂CH₂ Si(OCH₂ CH₃)₃,
decyltrichlorosilane,
dichloromethyl(4-methylphenethyl)silane,
diethoxymethylphenylsilane,
[3-(diethylamino)propyl]trimethoxysilane,
3-(dimethoxymethylsilyl)-1-propanethiol,
dimethoxymethylvinylsilane,
3-[tris(trimethylsilyloxy)silyl]propyl methacrylate,
trichloro[4-(chloromethyl)phenyl]silane,
methylbis(trimethylsilyloxy)vinylsilane,
methyltripropoxysilane, and
trichlorocyclopentylsilane.

The second reaction is the condensation polymerization of those monomers. Hydrolysis of trialkoxysilyl monomers is also straight forward in this context. Specifically, it involves the solubilization of those monomers followed by addition of an acid to a low pH in the presence of water. The acid can be sulfuric acid, glacial acetic acid, citric acid or hydrochloric acid.

A condensation polymerization approach may also be employed. For example, in the same pot, the pH may be raised to ˜8-12 with sodium hydroxide, ammonium, sodium bicarbonate, or an amine base to initiate the condensation polymerization of the hydroxy silane monomers. Alternatively, inorganic catalysts known to polymerize trialkoxysilanes can be employed (i.e., titanate and tin compounds). Fluorescent tags with hydroxy functionality such as Scopoletin, 4-methylumbelliferone, Fraxetin, Esculin, and Daphnetin can be added to the mixture at any time to condense with the forming oligomer at reactive hydroxy sites. After stirring for some time this polymerization is stopped by lowering the pH to neutral. At neutral pH, the polymer is stable and will not further polymerize or decompose through hydrolysis.

The resulting mixture can be solubilized in organic solvents miscible in water, preferably isopropyl alcohol in a ratio of 1:0.01 Water:IPA up to 0.01:1 Water:IPA, ideally between 25% and 75% IPA in water. Other solvents systems may be used as well depending on the hydrophobicity or hydrophilicity of the silane monomers used in the polymer formulation, the fluorescent tag used as well, and the composition of the intended tank partner. Alternatively, the material can be isolated through filtration, distillation, chromatography, and other methods known by those skilled in the art. The resulting material can be reconstituted in organic and aqueous solvents or mixtures of organic and aqueous solvents. Other formulations may take the form of wettable powders, dissolvable packets, wettable granule, pellets, dissolvable pills, inks, foams, concentrate solutions, or emulsion concentrates. These may be formulated with any solvent, dispersants, surfactants, polymers, anti-foam agents, coupling agents, small molecules, binders, resins, silane monomers, crosslinkers, surface modifiers or inks. These may be added to the formulation to increase adhesion, chemical durability, abrasion durability, and adhesive durability of the final coating.

While in the preferred embodiment the anti-viral compound and UV reflecting compound (or an antiviral compound modified to reflect UV light) is sprayed or dispensed as a foam, in still other embodiments the material is infused into a cloth or towelette such that it takes the form of a hand wipe for wiping down objects such as cellular phones, food, jars, bottles, telephones, remote controls, handles and the like. In some embodiments, the invention includes a towelette-dispensing box that dispenses one towelette or wipe at a time. In some embodiments, the traceable solution may be applied directly within the bins where vegetables, fruits, and other product is found in grocery stores. Such bins may be illuminated with UV so that the customer can see that the produce is protected by the traceable solution.

In an alternative embodiment, the UV antiviral compound is dispensed from a handheld sprayer, such as in a hair spray bottle or similar, which is either manually actuated or under positive pressure. In other embodiments, the material is provided with a gel such as hair gel. In that sense, the present embodiment relates additionally to hairspray, fabric softener, pesticides, fruit and vegetable preservatives, food preservatives, toilet bowl cleaner, any type of moisturizer, toothpaste, mouthwash, eyedrops, eardrops, cotton swabs of all types pre-treated with the UV traceable solution, all types of anti-fungal spray, all types of antifungal fogging solutions, furniture polish, condoms, plastic gloves of any kind, prophylactic covers, nasal sprays, window cleaners, furniture polish and dry cleaning solutions that non-exclusively use or employ sodium hypochlorite, hydrochloric acid, peg-2 hydrogenated tallow amine, alcohol ethoxylate, methyl salicylate and benzenesulfonic acid, tetrachloroethylene, perchloroethylene and/or trichloroethane.

In some embodiments, UV reflective polymeric binding agents are contemplated to conjugate with the above-described quinolone compounds. The polymeric binding agents provide a glue-like function to provide durable antimicrobial efficacy while maintaining the viscous structure of the spray. High molecular weight polymers can be dissolved or dispersed in water to form a well-distributed 3D network or uniform microsphere suspension. Those stretched polymer chains or massive dispersed microspheres provide broad surface areas to allow conjugate molecules to anchor onto them. The interactions between binding polymer affinitive groups/surfaces with conjugate molecules include but are not limited to van der Waals interaction, complex combination, ionic interaction, hydrogen bonds, crosslinking, free radical interaction, etc. The synergistic functions of binding agents and conjugates aqueous systems can extend their shelf-life, reduce chlorine odor, reduce gas phase corrosiveness and reduce toxicity.

In the foregoing description, various features of the disclosure are grouped together in limited disclosed embodiments for the purpose of streamlining the disclosure. This method of disclosure is not to be interpreted as reflecting an intention that the disclosure requires more features than are expressly recited in each embodiment. Rather, inventive aspects lie in less than all features of a single foregoing disclosed embodiment.

It is to be understood that the above-described arrangements are only illustrative of the application of the principles of the disclosure. Numerous modifications and alternative arrangements may be devised by those skilled in the art without departing from the spirit and scope of the disclosure and the appended claims are intended to cover such modifications and. arrangements. Thus, while the disclosure has been shown and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

Further to the above, the foregoing description of the preferred embodiment of the present invention has been presented for the purpose of illustration and description. It is not intended to be exhaustive or to limit the invention to the precise form disclosed. Many modifications and variations are possible in light of the above teachings. It is intended that the scope of the present invention to not be limited by this detailed description, but by the claims and the equivalents to the claims appended hereto.

What is claimed is:

1. A dispenser, the dispenser comprising:
a dispensing device containing a dispensing device solution, the dispensing device solution comprising C₂₀H₂₉NO₇Si;
the dispensing device solution having antiviral properties and reflecting or emitting UV light;
wherein the dispensing device solution may be used in combination with antimicrobial products;
wherein the dispensing device solution further comprises polymerizable UV absorbing and fluorescing elements, wherein the polymerizable UV absorbing and fluorescing elements are biodegradable, water soluble and copolymerize into hydrogel polymers;
the dispensing device further comprising a dispensing unit; and
a UV light source.

2. The dispenser of claim 1, wherein the polymerizable UV absorbing and fluorescing elements fluoresce in the presence of a UV light source.

3. The dispenser of claim 1, the dispensing device solution further comprising quinolone major product I., quinolone major product II., quinolone compound A., and/or quinolone compound B, Scopoletin, 4-methylumbelliferone, Fraxetin, Esculin, and Daphnetin.

4. The dispenser of claim 3, wherein the dispensing unit further comprises a cannister or hose providing an aerosolized spray in use, the cannister or hose being operably attached to the dispensing unit.

5. The dispenser of claim 3, wherein the antiviral disinfecting solution forms a self-assembled siloxane polymer and monomeric tag admixture in use.

6. The dispenser of claim 3, wherein the dispensing unit comprises a fogger, and wherein the dispensing device solution has antibacterial properties.

7. The dispenser of claim 1, wherein the dispensing device solution further comprises titanium dioxide, titanium dioxide providing the antibacterial properties of the dispensing device solution.

8. The dispenser of claim 3, wherein upon ejection from the dispenser the dispensing device solution forms a photoluminescent oligomer tag and self-assembled siloxane polymer admixture.

9. The dispenser of claim 7, wherein the antibacterial properties of titanium dioxide are induced by UV wavelengths in the range of 240 nm to 280 nm.

10. The dispenser of claim 7, wherein the antibacterial properties of titanium dioxide are provided by hydroxy radicals.

11. The dispenser of claim 1, wherein the dispensing unit solution further comprises Chlorine dioxide, Quaternary ammonium Citric acid, Thymol Dodecylbenzenesulfonic acid, Lactic acid, Ethyl alcohol, Quaternary Ammonium, Glycolic acid, Hydrochloric acid, Hydrogen peroxide, Hydrogen peroxide, Ammonium carbonate, Ammonium bicarbonate, Hydrogen peroxide, Octanoic acid, Peroxyacetic acid, Peroxyoctanoic acid, Peroxyacetic acid, Hydrogen peroxide, Silver Hypochloric acid, Hypochlorous acid, Isopropyl alcohol, Quaternary ammonium, L-Lactic acid, Lactic acid, Octanoic acid, Peracetic acid, Phenolic, and/or Ethanol.

12. The dispenser of claim 1, wherein the polymerizable UV absorbing and fluorescing elements are integrated into a lipidic solution.

13. The dispenser of claim 12, wherein the UV absorbing elements comprise Para Amino Benzoic Acid (PABA).

14. The dispenser of claim 12, wherein the UV absorbing elements further comprise Benzophenone-1.

15. The dispenser of claim 12, wherein the UV absorbing elements further comprise Benzotriazole.

16. The dispenser of claim 1, wherein the dispensing device solution further comprises Potassium peroxymonosulfate, Sodium chloride, Quaternary ammonium, Isopropanol Quaternary ammonium, Isopropanol, Glutaraldehyde, Sodium carbonate, Peroxyhydrate, Silver ion, Sodium chloride, Sodium chlorite, Sodium dichloroisocyanurate dihydrate, Sodium dichloro-S-triazinetrione, Sodium dichloroisocyanurate, Sodium hypochlorite, Sodium carbonate, Thymol, and/or Triethylene glycol.

17. An antiviral disinfecting solution, comprising:
a quinolone compound, the quinoline compound comprising quinolone major product I., quinolone major product II., quinolone compound A., and/or quinolone compound B.;
wherein the quinolone compound reflects UV light;
wherein the antiviral disinfecting solution further comprises polymerizable UV absorbing and fluorescing elements;
wherein the antiviral disinfecting solution has antibacterial properties;
wherein the polymerizable UV absorbing and fluorescing elements are biodegradable, water soluble and copolymerize into hydrogel polymers; and
wherein the antiviral disinfecting compound may be used in combination with antimicrobial products.

18. The antiviral disinfecting solution of claim 17, wherein benzotriazoles are used to copolymerize the UV absorbing and fluorescing elements into hydrogel polymers, and wherein titanium dioxide provides the antibacterial mechanism for the antibacterial properties of the antiviral disinfecting solution.

19. The antiviral disinfecting solution of claim 17, wherein in use the antiviral disinfecting solution forms an photoluminescent terminating group and self-assembled siloxane polymer admixture.

20. The antiviral disinfecting solution of claim 17, wherein the UV absorbing elements comprise Para Amino Benzoic Acid (PABA), Benzophenone-1, and/or Benzotriazole.

21. An antiviral disinfecting solution comprising:
at least one quinolone compound, wherein the quinolone compound emits or reflects UV light;
the at least one quinolone compound, the quinolone compound comprising quinolone scaffold compound I., quinolone scaffold compound II., quinolone compound A., and/or quinolone compound B;
wherein the antiviral disinfecting compound further comprises polymerizable UV absorbing and fluorescing elements, wherein benzotriazoles are used to copolymerize the UV absorbing and fluorescing elements into hydrogel polymers;
wherein the antiviral disinfecting compound has antibacterial properties and wherein titanium dioxide provides the antibacterial mechanism for the antibacterial properties;
wherein the UV absorbing and fluorescing elements comprise 2-phenyl benzotriazole having a polymerizable acrylic group;
wherein the polymerizable UV absorbing and fluorescing elements are biodegradable, water soluble and copolymerize into hydrogel polymers;
wherein the antiviral disinfecting compound may be used in combination with microbial products; and
wherein the synthetic route to the antiviral disinfecting solution includes the bonding of two different UV absorbing compounds having different UV absorbing spectra.
